(19) United States
(12) Patent Application Publication
(10) Pub. No.: US 2026/0345879 A1
(43) Pub. Date: Nov. 5, 2026

(54) BRESTFEEDING DEVICE

(71) Applicant: CLOSER HEALTHCARE LTD, Tel Aviv (IL)

(72) Inventors: Shada ABU RAS, Ilut (IL); Moataz BIADCY, Tel Aviv (IL)

(21) Appl. No.: 19/102,647

(22) PCT Filed: Aug. 28, 2023
(86) PCT No.: PCT/IL2023/050910

§ 371 Date: Feb. 10, 2025

(30) Foreign Application Priority Data
Aug. 28, 2022 (IL) 296005

(51) Int. Cl. A61M 1/06

(52) U.S. Cl. CPC A61M 1/068; A61M 2205/3334

(57) ABSTRACT

The present disclosure provides a device for assisting in real-time breastfeeding of an infant. The device is particularly relevant for breastfeeding infants who experience difficulties in natural breastfeeding, e.g. due to lack of capability for applying sufficient suction force on the nipple to suction the necessary portion of breastmilk, such as in the case of a preemie. The device is making use of a pump that is configured to induce suctioning conditions on a nipple, e.g. by being in fluid communication with a suction portion of the device that is designed to be fitted over a nipple-including portion of the breast of the subject. The pump is also in fluid communication with a nipple-like portion of the device, which is a part that emulates the natural nipple such that the infant is intended to fit his/her mouth over it and apply suction conditions on it. The pump is configured to operate in a two-phases cycle, wherein in the first phase of the cycle the pump suctions breastmilk from the subject and in the second phase of the cycle the suctioned breastmilk is pumped towards the nipple-like portion into the mouth of the infant. The amount of suctioned breastmilk and the flow rate of breastmilk to the infant can be determined by external outputs or objective sensed data, e.g. a pressure sensor that senses the suction forces of the infant and in response the pump is tuned to provide the required pumping assistance to ensure the delivery of the right amount of breastmilk to the infant.

### TECHNOLOGICAL FIELD

The present disclosure is in the field of real-time breastfeeding assisting device.

### BACKGROUND ART

References considered to be relevant as background to the presently disclosed subject matter are listed below:
US2016256618
CN108992720
US2021393861
US2021361837
CN216571014
CN212262015
CN209575378
US2014052056

Acknowledgement of the above references herein is not to be inferred as meaning that these are in any way relevant to the patentability of the presently disclosed subject matter.

### GENERAL DESCRIPTION

The present disclosure provides a device for assisting in real-time breastfeeding of an infant. The device is particularly relevant for breastfeeding infants who experience difficulties in natural breastfeeding, e.g. due to lack of capability for applying sufficient suction force on the nipple to suction the necessary portion of breastmilk, such as in the case of a preemie.

The device is making use of a pump that is configured to induce suctioning conditions on a nipple, e.g. by being in fluid communication with a suction portion of the device that is designed to be fitted over a nipple-including portion of the breast of the subject. The pump is also in fluid communication with a nipple-like portion of the device, which is a part that emulates the natural nipple such that the infant is intended to fit his/her mouth over it and apply suction conditions on it. The pump is configured to operate in a two-phases cycle, wherein in the first phase of the cycle the pump suctions breastmilk from the subject and in the second phase of the cycle the suctioned breastmilk is pumped towards the nipple-like portion into the mouth of the infant. The amount of suctioned breastmilk and the flow rate of breastmilk to the infant can be determined by external outputs or objective sensed data, e.g. a pressure sensor that senses the suction forces of the infant and in response the pump is tuned to provide the required pumping assistance to ensure the delivery of the right amount of breastmilk to the infant. Therefore, the device provides a solution that assists in real-time breastfeeding and maintaining the attachment between the infant and the breastfeeding mother. The attachment is important for biological, emotional, mental, and social infant's development. Instinctual attachment behaviors in the baby are activated by cues or signals from the caregiver and is defined as a “mutual regulatory system” in which the baby and the caregiver influence one another over time. Breastfeeding is the first attachment between infant and mother, as it creates a bonding experience because it promotes skin-to-skin contact, more holding and stroking. When women breastfeed and feel that attachment, dopamine (a hormone associated with reward) levels decrease for prolactin (milk producing hormone) levels to rise.

Thus, a first aspect of the present disclosure provides a device, having a general shape of a breast cup designed to be fitted on a breast, for assisting in real-time breastfeeding of an infant. The device comprises a suction space for receiving a nipple. The suction space may be defined by a certain contour of a part of the device that is suitable to be fitted over a nipple to allow application of suction conditions on the nipple. The device further comprising a pump in fluid communication with the suction space and is configured to induce suction conditions (suction conditions mean conditions of pressure difference sufficient for causing suction of milk from the breast of the woman) in the suction space for suctioning breastmilk from the nipple placed within the suction space. The term “suction condition” means conditions of pressure difference sufficient for causing suction of milk from the breast of the woman. The device further comprising a nipple-like portion, at least part of it extending away from the breast, for allowing suction by an infant that induces suction conditions within the suction space. Namely, the nipple-like portion emulates a human nipple such that the infant would try to suction from it. The pump is in fluid communication with the nipple-like portion for allowing suctioned breastmilk from the nipple to be pumped to the nipple-like portion at a desired pressure and/or amount.

It is to be noted that any combination of the described embodiments with respect to any aspect of this present disclosure is applicable. In other words, any aspect of the present disclosure can be defined by any combination of the described embodiments.

In some embodiments of the device, the pump is configured to perform a suction action from the nipple and pumping action of the suctioned breastmilk to the nipple-like portion in a single, two-phased, cycle.

In some embodiments of the device, the nipple-like portion comprises a pressure sensor for sensing the suction intensity of the infant, namely the pressure difference that the infant produces when he/she are suctioning the nipple-like portion, so as to generate suction sensed data indicative of the suction characteristics of the infant to be used for controlling the suction and/or pumping force of the pump to compensate the deficiency in the force of the infant, thereby providing the desired breastmilk flowrate to the infant. It is to be noted that by controlling the suction and/or pumping force, the amount of milk and its flow rate to the infant are defined.

In some embodiments, the device further comprising a processing circuitry in data communication with the pump for controlling the pumping profile thereof, namely the intensity of the pumping/suction and/or the duration of each phase or cycle, etc., and configured for receiving said suction sensed data to set the pumping profile of the pump to obtain the desired pressure and/or amount of breastmilk to be pumped to the infant.

In some embodiments, the device further comprising an internal layer, which a portion thereof defines the suction space and an external layer, which a portion thereof defines the nipple-like portion, wherein the pump is housed between the internal and the external layers.

In some embodiments of the device, the suction space is defined by a nipple-receiving portion. The nipple-receiving portion is a portion of the device that has a contour that fits the contour of the breast area that comprises the nipple of the subject.

In some embodiments of the device, the nipple-receiving portion is defined by a portion of the internal layer.

In some embodiments of the device, the internal layer and the external layer are attached to one another, defining an internal volume. The pump is housed in said internal volume.

In some embodiments of the device, the internal volume is sealed.

In some embodiments, the device comprising a receptacle fluidically connected to the pump for accumulating the suctioned breastmilk up to a desired amount and upon accumulating the desired amount, allowing the accumulated amount to be pumped, namely delivered, to the infant.

In some embodiments of the device. the receptacle comprises sensing means for sensing the amount of accumulated breast milk within the receptacle and generate accumulated breastmilk sensed data based on that. Upon sensing that a desired amount is accumulated, the sensing means send signals either to the pump, a processing circuitry, directly to valves, or to any combination thereof to allow the accumulated breastmilk to be pumped and delivered to the infant.

In some embodiments of the device, the receptacle is being a part of the pump.

In some embodiments of the device, the receptacle is located on a first flow path defined between the suction space and the pump, when breastmilk is suctioned into the receptacle, and on a second flow path defined between the pump and the nipple-like portion, when the breast milk is delivered from the receptacle to the nipple-like portion.

In some embodiments, the device further comprising a first valve switchable between open and closed states and is configured to open and close the flow communication between the suction space and the pump, and a second valve switchable between open and closed states and is configured to open and close the flow communication between the pump and the nipple-like portion. At a first, accumulating, mode of operation, the first valve is open, and the second valve is closed when the accumulated breastmilk in the receptacle is less than a certain threshold, thereby allowing flow of breastmilk from the suction space to the pump and/or the receptacle. At a second, pumping, mode of operation, the first valve is closed, and the second valve is open when the accumulated breastmilk in the receptacle is equal or higher than a certain threshold, thereby allowing flow of breastmilk from the pump and/or the receptacle to the nipple-like portion. The threshold is defined according to the desired amount, which can be set prior to the operation of the device.

In some embodiments of the device, the first valve is located between an inlet of the receptacle and the suction space, and the second valve is located between an outlet of the receptacle and the nipple-like portion.

In some embodiments, the device further comprising a processing circuitry, which can be the same or different than the processing circuitry that is in data communication with the pump, in data communication with the first and second valves for controlling their states based on an indication of the accumulated breastmilk in the receptacle. Namely, the processing circuitry may receive the accumulated breastmilk sensed data and set the states of the first and second valves accordingly.

In some embodiments, the device further comprising a first valve configured to open and close the flow communication between the suction space and the pump, and a second valve configured to open and close the flow communication between the pump and the nipple-like portion, wherein (i) at a first phase of the suction cycle of the pump, the first valve is open and the second valve is closed, and (ii) at a second phase of the cycle, the first valve is closed and the second valve is open.

In some embodiments of the device, the nipple-like portion comprise an aperture at a tip thereof to allow delivery of breastmilk to the infant. The aperture is in fluid communication with the pump via pipes or tubes.

In some embodiments, the device further comprising a button for activate and disactivate the operation of the device.

Yet another aspect of the present disclosure provides a method for assisting a real-time breastfeeding of an infant. The method comprising: (i) applying suctioning conditions on a nipple of a subject to suction breastmilk; (ii) accumulating a desired amount of breastmilk; and (iii) pumping the accumulated breastmilk towards a mouth of an infant. Steps (i)-(iii) of the method are carried out in continuous and repeating manner, namely breastmilk is suctioned from the nipple of the subject until a desired accumulated amount of breastmilk is obtained, which is then pumped towards the mouth of the infant and then a new cycle of suctioning, accumulation and pumping begins.

In some embodiments of the method, steps (i)-(ii) are carried out in a first phase of a single operation cycle of a pump and (iii) is carried out in a second phase of a single operation cycle of said pump. Namely, in one cycle of operation of the pump, the breastmilk is suctioned from the nipple and then pumped to the infant to experience real-time breastfeeding.

In some embodiments, the method further comprising sensing suction force applied by the infant on a nipple-like portion to generate suction force sensed data, said pumping comprises adjusting the pumping intensity profile based on the suction force sensed data to deliver a desired flow rate of breastmilk to the infant via said nipple-like portion.

In some embodiments, the method further comprising receiving input data indicative of the desired flow rate profile of breastmilk to the infant, wherein (i)-(iii) are adjusted based on said input data. Namely, each one of the following can be adjusted independently according to the input data: (i) the suction forces applied on the nipple of the subject; (ii) the amount of milk to be accumulated before being pumped; and (iii) the pumping intensity of the accumulated breastmilk towards the mouth of the infant.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C are schematic illustrations of a cross-sectional view of a non-limiting example of the device and the processes carried out thereby according to the present disclosure. FIG. 1A is a clean cross-sectional view; FIG. 1B demonstrates the suction step including suction of breastmilk to the pump; FIG. 1C demonstrates the pumping step including pumping of breastmilk from the pump to the nipple-like portion.

FIGS. 2A-2C are schematic illustrations of a non-limiting example of an embodiment of the device according to an aspect of the present disclosure.

### DETAILED DESCRIPTION

The following figures are provided to exemplify embodiments and realization of the invention of the present disclosure.

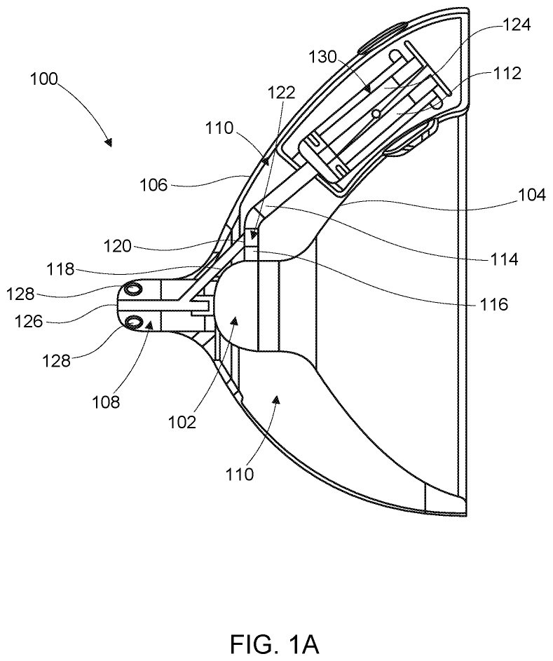

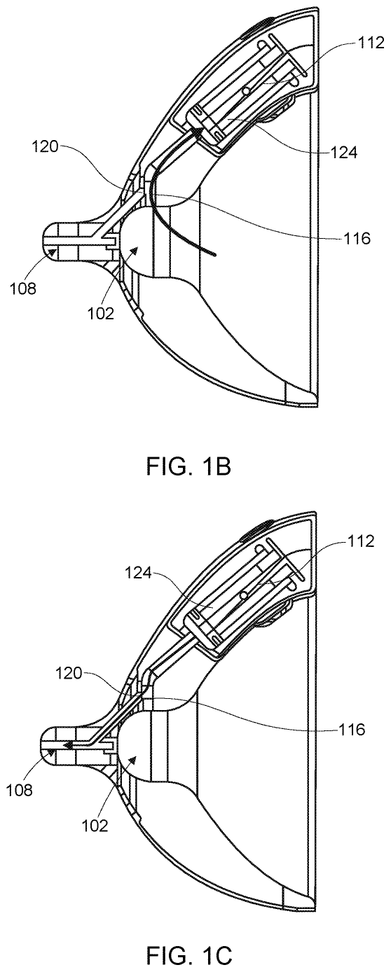

Reference is made to FIGS. 1A-1C, which are schematic illustrations of a cross-sectional view exemplifying a non-limiting embodiment of the breastfeeding device and the breastfeeding process being carried out by it.

The device 100 is designed to be suitable for placing over a breast of a breastfeeding subject, namely the device has a generally breast cup shape. The device 100 includes a suction space 102 defined by a cup shape contour of an internal layer 104 of the device. The internal layer 104 can be made of any biocompatible materials, such as plastic and/or silicon. The suction space 102 is designed to receive the breast of the breastfeeding subject such that the nipple of the subject is exposed to induction of suction condition applied by the pump of the device, as is explained below.

An external layer 106 of the device, that can be made of plastic for example, defines the contour of a nipple-like portion 108 that is designed to be received in a mouth of an infant for allowing the infant to suction the nipple-like portion 108 and consume breastmilk that flows outwards from the nipple-like portion 108.

It is to be noted that the terms “internal” and “external” with respect to the layers are made with respect to the proximity to the breast of the subject when wearing the device, namely the internal layer is the layer that comes into contact, at least partially, with the breast and the external layer faces outwardly from the breast.

The internal layer 104 and the external layer 106 are attached one to the other so they confine an internal volume 110 therebetween. A pump 112 is housed within the internal volume 110 and is in fluid communication with the suction space 102 and the nipple-like portion 108. The fluid communication to the suction space 102 is made by a first tube 114. The first tube 114 comprises a first valve 116 for controllably blocking and opening the fluid communication between the suction space 102 and the pump 112. The fluid communication to the nipple-like portion 108 is made by a second tube 118 that is bifurcated from the first tube 114 at a portion of the first tube 114 between the first valve 116 and the pump 112. A second valve 120 is disposed at or at the proximity of the bifurcation point 122 along the second tube 118 for controllably blocking and opening the fluid communication between the nipple-like portion 108 and the pump 112.

The pump 112 is configured to induce suction conditions in the suction space 102 to pump breastmilk from the nipple of the subject towards a receptacle/reservoir 124 of the pump 112, which is exemplified by the arrow in FIG. 1B. The application of suction conditions by the pump 112 is made during a first phase of the operational cycle of the pump 112, while the second valve 120 is in blocking state, not allowing breastmilk to flow to the nipple-like portion 108, and the first valve 116 is in open state, allowing breastmilk to flow between the suction space 102 and the receptacle 124 of the pump 112.

Once the receptacle 124 is sufficiently filled with breastmilk, namely that a desired amount of breastmilk is accumulated therein, the pump 112 pumps the breastmilk from the receptacle 124 towards the nipple-like portion 108 to be discharged from an aperture 126 formed therein and to be consumed by the infant, which is exemplified by the arrow in FIG. 1C. The pumping action is performed during a second phase of the operational cycle of the pump 112, while the second valve 120 is in open state, allowing breastmilk to flow to the nipple-like portion 108, and the first valve 116 is in blocking state, not allowing breastmilk to flow between the suction space 102 and the receptacle 124 of the pump 112.

A pressure sensor 128 is disposed at the nipple-like portion 108 and is configured to sense the suction force applied by the infant on the nipple-like portion 108. The suction of the baby causes flow of the breastmilk from the receptacle 124 towards the nipple-like portion 108 to be discharged from the aperture 126 to the mouth of the infant. The device 100 is configured to process the sensed force of the suction of the infant and adjust the pumping force of the pump 112 so as to compensate the lack of suctioning force of the infant, thereby resulting in the desired amount of breastmilk that is received by the infant.

The operation of the device is controlled by a processing circuitry 130, i.e. a controller, control unit, processor. The processing circuitry 130 can be housed at any portion of the device, though typically it is housed within the internal volume 110 or is being part of the pump 112. The processing circuitry 130 is configured to control the following: (i) the suction intensity applied on the suction space during the first phase of the pump; (ii) the pumping intensity of the accumulated breastmilk in the receptacle towards the nipple-like portion; (iii) the state of the first valve; and (iv) the state of the second valve. The processing circuitry 130 is configured to receive an input of breastmilk consumption data indicative of the amount of any one of: (i) the amount of breastmilk the infant is required to consume during a breastfeeding session; (ii) the flow rate profile of breastmilk to be supplied to the infant; and (iii) a combination thereof. The data can be derived from input data of the age of the infant, medical-related information of the infant or any other relevant data that may contribute to the adjustment of the operation of the pump by the processing circuitry. The data can be inputted via an API through a smartphone or via a dedicated user interface.

The processing circuitry 130 is further configured to receive the sensed data from the pressure sensor 128 and to adjust the pumping force of the pump 112 to compensate the lack of suction force applied by the infant on the nipple-like portion 108 so as to obtain the desired flow rate profile of breastmilk according to the input data.

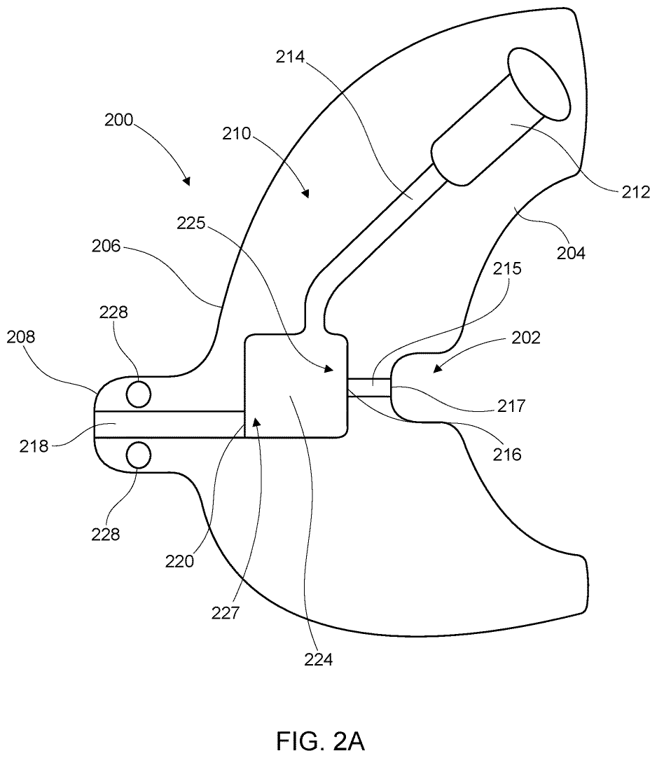

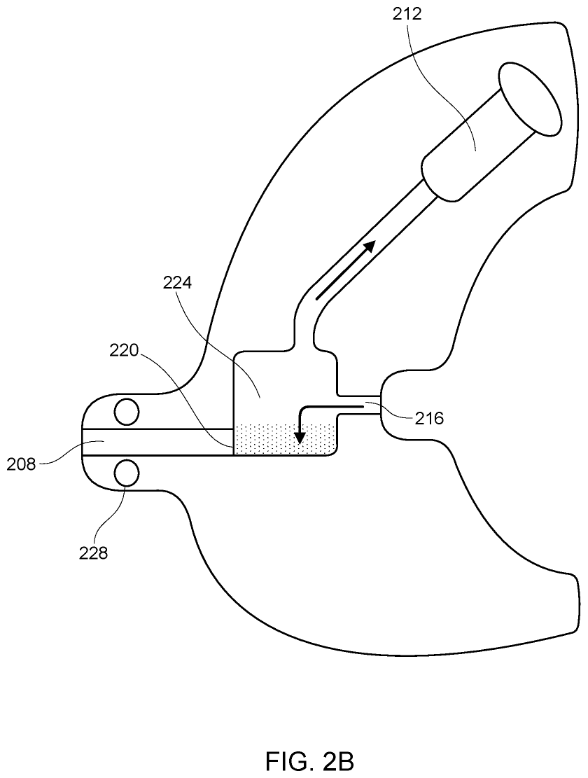

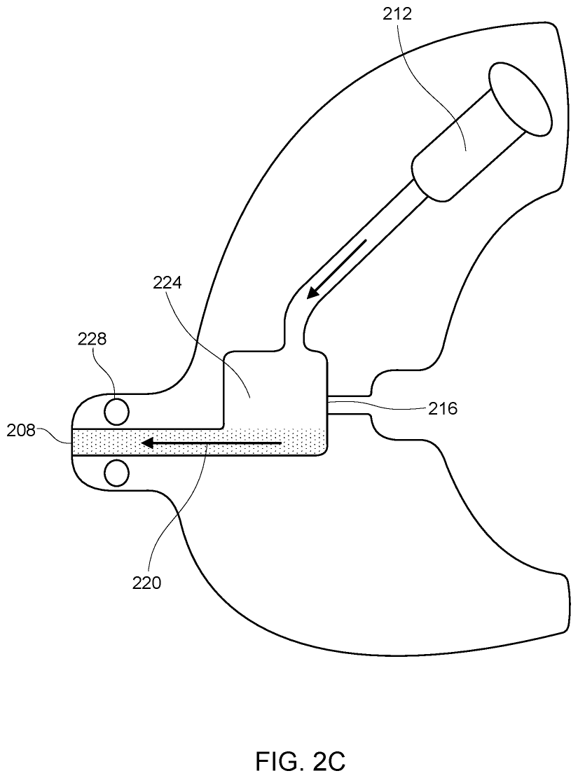

Reference is now being made to FIGS. 2A-2C, which are schematic illustrations of a non-limiting example of an embodiment of the device according to an aspect of the present disclosure. These figures exemplify the accumulation of breastmilk within a receptacle of the device and controlled delivery of the accumulated breastmilk to the infant. The device 200 is designed to be suitable for placing over a breast of a breastfeeding subject, namely the device has a generally breast cup shape. The device 200 includes a suction space 202 defined by a cup shape contour of an internal, proximal layer/surface 204 of the device. The internal surface 204 can be made of any biocompatible materials, such as plastic and/or silicon. The suction space 202 is designed to receive the breast of the breastfeeding subject such that the nipple of the subject is exposed to induction of suction condition applied by the pump of the device, as is explained below.

An external, distal surface 206 of the device, that can be made of plastic for example, defines the contour of a nipple-like portion 208 that is designed to be received in a mouth of an infant for allowing the infant to suction the nipple-like portion 208 and consume breastmilk that flows outwards from the nipple-like portion 208.

It is to be noted that the terms “proximal” and “distal” with respect to the layers are made with respect to the proximity to the breast of the subject when wearing the device, namely the proximal layer is the layer that comes into contact, at least partially, with the breast and the distal layer faces outwardly from the breast and contains the nipple-like portion that is received in the mouth of the infant.

The proximal layer 204 and the distal layer 206 are attached one to the other so they confine an internal volume 210 therebetween. A pump 212 is housed within the internal volume 210 and is in fluid communication with the suction space 202 and the nipple-like portion 208. The fluid communication to the suction space 202 is made by a first tube 214 that couples the pump 212 and a breastmilk receptacle 224 and a third tube 215 that couples the receptable 224 with to an aperture of a nipple-receiving portion 217 that constitutes a part of the suction space 202. The receptacle 224 is disposed in the internal volume 210, namely it is confined between the proximal layer 204 and the distal layer 206. A first valve 216 is disposed between the receptacle 224 and the nipple-receiving portion 217 to controllably blocking and opening the fluid communication between the suction space 202 and the pump 212. When the first valve 216 is open and the pump 212 operates, suction conditions apply on the suction space 202 to suction breastmilk from a nipple of a subject that is received in the nipple-receiving potion 217. The milk that is suctioned flows into the receptacle 224 through a first opening 225 and accumulates there. The fluid communication between the pump 212 and the nipple-like portion 208 is made by the first tube 214 that links it to the receptacle 224 and a second tube 218 that couples the receptacle to the nipple-like portion 208. A second valve 220 is disposed between the receptacle 224 and the nipple-like portion 208 for controllably blocking and opening the fluid communication between the nipple-like portion 208 and the pump 212. When the second valve 220 is open and the pump 212 operates, breastmilk that is accumulated in the receptacle 224 flows from the receptacle 224 through a second opening 227 towards the nipple-like portion to be delivered to an infant.

Pressure sensors 228 are disposed at the nipple-like portion 208 and are configured to sense the suction force applied by the infant on the nipple-like portion 208. Upon sensing pressure on the pressure sensors 228 applied by the suction of the infant of the nipple-like portion 208, the pump is triggered to start pumping. At the first phase, the first valve 216 is in an open state allowing the suction of breastmilk from the nipple of the subject into the receptacle 224, while the second valve 220 is in a blocking state, blocking the flow of the breastmilk from the receptacle 224 towards the nipple-like portion 208, thereby allowing accumulation of breastmilk in the receptacle 224. This is exemplified in FIG. 2B. Once breastmilk is accumulated to a desired amount, the first valve is switched to the blocking state and the second valve 220 is switched to the open state, thereby allowing flow of the accumulated breastmilk from the receptacle 224 to the nipple-like portion 208 to be delivered to the infant. This is exemplified by FIG. 2C. This process can be repeated as long as the infant apply suction conditions on the pressure sensors 228.

The operation of the device is controlled by a processing circuitry (not shown), i.e. a controller, control unit, processor. The processing circuitry can be housed at any portion of the device, though typically it is housed within the internal volume 210 or is being part of the pump 212. The processing circuitry is configured to control the following: (i) the suction intensity applied on the suction space during the first phase of the pump; (ii) the pumping intensity of the accumulated breastmilk in the receptacle towards the nipple-like portion; (iii) the state of the first valve; (iv) the state of the second valve; and (v) the adjustment of the accumulation threshold of the breastmilk in the receptacle. The processing circuitry is configured to receive an input of breastmilk consumption data indicative of the amount of any one of: (i) the amount of breastmilk the infant is required to consume during a breastfeeding session; (ii) the flow rate profile of breastmilk to be supplied to the infant; and (iii) a combination thereof. The data can be derived from input data of the age of the infant, medical-related information of the infant or any other relevant data that may contribute to the adjustment of the operation of the pump by the processing circuitry. The data can be inputted via an API through a smartphone or via a dedicated user interface.

The processing circuitry is further configured to receive the sensed data from the pressure sensor 228 and to adjust the pumping force of the pump 212 to compensate the lack of suction force applied by the infant on the nipple-like portion 208 so as to obtain the desired flow rate profile of breastmilk according to the input data.

1-21. (canceled)

22. A device in the form of a breast cup, configured to be fitted on a breast, for assisting in real-time breastfeeding of an infant, comprising:
a suction space for receiving a nipple;
a pump in fluid communication with the suction space and is configured to induce suction conditions in the suction space for suctioning breastmilk from the nipple placed within the suction space;
a nipple-like portion for allowing suction of an infant;
wherein the pump is in fluid communication with the nipple-like portion for allowing suctioned breastmilk from the nipple to be pumped to the nipple-like portion at a desired pressure and/or amount.

23. The device of claim 22, wherein the pump is configured to perform a suction action from the nipple and pumping action of the suctioned breastmilk to the nipple-like portion in a single, two-phased, cycle.

24. The device of claim 22, wherein the nipple-like portion comprises a pressure sensor for sensing the suction intensity of the infant so as to generate suction sensed data indicative of the suction characteristics of the infant to be used for controlling the suction and/or pumping force of the pump to compensate the deficiency in the force of the infant, thereby providing the desired breastmilk flowrate to the infant.

25. The device of claim 24, comprising a processing circuitry in data communication with the pump for controlling the pumping profile thereof and configured for receiving said suction sensed data to set the pumping profile of the pump to obtain the desired pressure and/or amount of breastmilk to be pumped to the infant.

26. The device of claim 22, comprising an internal layer having a part defining the suction space and an external layer having a part defining the nipple-like portion, wherein the pump is housed between the internal and the external layers.

27. The device of claim 26, wherein the internal layer and the external layer are attached to one another, defining an internal volume, said pump is housed in said internal volume.

28. The device of claim 27, wherein said internal volume is sealed.

29. The device of claim 22, comprising a receptacle fluidically connected to the pump for accumulating the suctioned breastmilk up to a desired amount and upon accumulating the desired amount, allowing the accumulated amount to be pumped to the infant;
wherein the receptacle comprises sensing means for sensing the amount of accumulated breast milk within the receptacle and generate accumulated breastmilk sensed data based on that, and upon sensing that a desired amount is accumulated, sending signals to allow the accumulated breastmilk to be delivered to the nipple-like portion.

30. The device of claim 29, wherein the receptacle is a part of the pump.

31. The device of claim 29, wherein the receptacle is located on a first flow path defined between the suction space and the pump, when breastmilk is suctioned into the receptacle, and on a second flow path defined between the pump and the nipple-like portion, when the breast milk is delivered from the receptacle to the nipple-like portion.

32. The device of claim 29, comprising a first valve switchable between open and closed states and is configured to open and close the flow communication between the suction space and the pump, and a second valve switchable between open and closed states and is configured to open and close the flow communication between the pump and the nipple-like portion, wherein (i) the first valve is open and the second valve is closed when the accumulated breastmilk in the receptacle is less than a certain threshold, and (ii) wherein the first valve is closed and the second valve is open when the accumulated breastmilk in the receptacle is equal or higher than a certain threshold.

33. The device of claim 32, comprising a processing circuitry in data communication with the first and second valves for controlling their states based on an indication of the accumulated breastmilk in the receptacle.

34. The device of claim 22, comprising a first valve configured to open and close the flow communication between the suction space and the pump, and a second valve configured to open and close the flow communication between the pump and the nipple-like portion, wherein (i) at a first phase of the suction cycle of the pump, the first valve is open and the second valve is closed, and (ii) at a second phase of the cycle, the first valve is closed and the second valve is open.

35. The device of claim 34, wherein the first valve is located between an inlet of the receptacle and the suction space, and the second valve is located between an outlet of the receptacle and the nipple-like portion.

36. The device of claim 22, wherein the nipple-like portion comprise an aperture at a tip thereof to allow discharge of breastmilk to the infant.

37. The device of claim 22, comprising a button for activate and disactivate the operation of the device.

38. A method for assisting a real-time breastfeeding of an infant, comprising:
(i) applying suctioning conditions on a nipple of a subject to suction breastmilk;
(ii) accumulating a desired amount of breastmilk;
(iii) pumping the accumulated breastmilk towards a mouth of an infant;
wherein (i)-(iii) are carried out in continuous and repeating manner.

39. The method of claim 38, wherein (i)-(ii) are carried out in a first phase of a single operation cycle of a pump and (iii) is carried out in a second phase of a single operation cycle of said pump.

40. The method of claim 38, comprising sensing suction force applied by the infant on a nipple-like portion to generate suction force sensed data, said pumping comprises adjusting the pumping intensity profile based on the suction force sensed data to deliver a desired flow rate of breastmilk to the infant via said nipple-like portion.

41. The method of claim 38, comprising receiving input data indicative of the desired flow rate profile of breastmilk to the infant, wherein (i)-(iii) are adjusted based on said input data.
